(19) United States
(12) Patent Application Publication
(10) Pub. No.: US 2011/0281127 A1
(43) Pub. Date: Nov. 17, 2011

(54) PREPARATION METHOD OF HYDROXYAPATITE COATING LAYER USING AEROSOL DEPOSITION AND HYDROTHERMAL TREATMENT, AND NANOSTRUCTURED HYDROXYAPATITE COATING LAYER PREPARED BY THE METHOD

(75) Inventors: Byung-Dong HAHN, Gyeongsangnam-do (KR); Dong-Soo Park, Gyeongsangnam-do (KR)

(73) Assignee: Korea Institute of Machinery and Materials, Daejeon (KR)

(21) Appl. No.: 12/871,434

(22) Filed: Aug. 30, 2010

(30) Foreign Application Priority Data
May 13, 2010 (KR) 10-2010-0044994

(51) Int. Cl. B32B 15/04; A61L 27/32

(52) U.S. Cl. 428/469; 427/2.27

(57) ABSTRACT

A method of preparing a nano-structured hydroxyapatite [Ca₁₀(PO₄)₆(OH)₂, HA] coating layer with improved biocompatibility is provided. The preparation method includes: (S1) putting hydroxyapatite [Ca₁₀(PO₄)₆(OH)₂, HA] powder in a powder chamber, placing a metal substrate in a deposition chamber, and keeping the chamber in vacuum state by using a vacuum pump; (S2) injecting carrier gas into the powder chamber and mixing with hydroxyapatite powder; (S3) spraying the mixture of the powder and the carrier gas onto the surface of metal substrate in the deposition chamber through a nozzle and depositing hydroxyapatite coating layer on the metal substrate; (S4) hydrothermal-treating the deposited hydroxyapatite coating layer; and (S5) rinsing the hydrothermal-treated hydroxyapatite coating layer and drying the layer. Additionally, a thermal-treated, nano-structured hydroxyapatite [Ca₁₀(PO₄)₆(OH)₂, HA] coating layer with improved biocompatibility fabricated by the above preparation method is provided.

### CROSS-REFERENCE TO RELATED APPLICATIONS

This application claims priority from Korean Patent Application No. 10-2010-0044994, filed on May 13, 2010, in the Korean Intellectual Property Office, the disclosure of which is incorporated herein by reference in its entirety.

### BACKGROUND OF THE INVENTION

1. Field of the Invention

Methods and layers consistent with what is disclosed herein relate to a preparation method of hydroxyaptite coating layer using aerosol deposition and hydrothermal treatment, and a nanostructured hydroxyapatite coating layer with superior biocompatibility prepared by the method.

2. Description of the Related Art

Titanium or titanium alloy is representative bio metal materials used in dental and medicinal field and orthopedics for many years, but titanium itself has shortcomings such as lack of bioactivity, subsequently long period of time until bone formation and low adhesion of osseous tissue.

To resolve these shortcomings, many studies have been conducted to improve the reactions and adhesion of osseous tissues by treating the surface of metal implant. Especially, when calcium phosphate ceramics like hydroxyapatite [Ca₁₀(PO₄)₆(OH)₂] with superior bioactivity and biocompatibility is used in a coated form on the surface of the metal implant, superior bio-applicability of ceramics and mechanical characteristics of metal can be both achieved. To be specific, hydroxyapatite is calcium phosphate ceramic and main component of human bone and teeth. Hydroxyapatite is widely used as a bone replacement in the dental and medicinal fields or orthopedics as it has superior osteoacusis effect, bioactivity, bio-applicability, protein adhesion, heavy metal adhesion and antibacterial performance.

Various methods of coating bioactivity ceramics like hydroxyapatite in the metal implant have been developed. Among these, the most commonly used coating method for now is plasma spray. However, this method requires the high temperature above 10,000° C., causing problems of phase separation during coating process, crack within the coating layer, low density of coating layer, low adhesion caused by oxidation between the interface of coated materials and the metal substrate, etc, thereby declining the durability and stability of implant.

To solve these problems, various methods of making hydroxyapatite coating layer with high density and superior adhesion to the metal substrate have been developed and recently, aerosol deposition (AD) has newly been developed in which a highly-dense ceramic coating layer is formed on various substrates like ceramics, metal and polymer at room temperature. AD method is to spray particles in solid phase onto a substrate in vacuum chamber. Superior adhesion between the substrate and coating layer is made by the strong collision of particles of coating materials against the substrate. The composition of the coating layer remains the same as that of starting particles because the deposition occurs at room temperature. Therefore, AD is an ideal method to produce hydroxyapatite coating layer whose quality is heavily dependent on the control of composition.

The microstructural feature of a hydroxyapatite coating layer by AD is that coating layer is composed of nano crystalline hydroxyapatite grain and amorphous phase. In AD process, the coated grains are fractured into the nano size due to the impact of high energy of grains toward substrate. Nano-sized hydroxyapatite in coating layer is very similar to the nano crystal of apatite found in natural bone in its size and crystal structure. Therefore, the method of nano-structured hydroxyapatite coating by AD is very appropriate in implant. Further, nano-structured hydroxyapatite ceramics has more superior biological effect because it has osteoblast function originated from its wide effective surface area and surface nano-roughness.

The crystallinity of coating layer is known to be an important factor to affect the biological features of hydroxyapatite coating. Amorphous hydroxyapatite is more soluble in body fluid and this dissolution rate drops the long-term stability of implant. Also, according to Q. Hu, Z. Tan, Y. Liu, J. Tao, Y. Cai, M. Zhang, H. Pan, X. Xu, and R. Tang, “Effect of Crystallinity of Calcium Phosphate Nanoparticles on Adhesion, Proliferation, and Differentiation of Bone Marrow Mesenchymal Stem Cells,” J. Mater. Chem., 17 4690-4698 (2007), in coating similar-sized particles of hydroxyapatite, crystallized hydroxyapatite coating is better than amorphous one in absorbing and proliferating the cells. Therefore, to improve the crystallinity of hydroxyapatite coating, the method of heating the coating after deposition is used. Traditionally, the heating is processed in a furnace to improve the crystallinity of hydroxyapatite coating layer by AD. However, when the temperature is above 500° C., the size of grains increases and this drops the biocompatibility drastically.

In this regards, we studied and completed a method for improving the crystallinity of coating layers on the substrate while inhibiting the growth of the grains.

### SUMMARY OF THE INVENTION

Exemplary embodiments overcome the above disadvantages and other disadvantages not described above. Also, the embodiments are not required to overcome the disadvantages described above, and an exemplary embodiment of the present invention may not overcome any of the problems described above.

To achieve the above object, in one embodiment, a method of preparing a nano-structured hydroxyapatite coating layer with improved biocompatibility may include: (S1) putting hydroxyapatite [Ca₁₀(PO₄)₆(OH)₂, HA] powder in a powder chamber, placing a metal substrate in a deposition chamber, and keeping the chamber in vacuum state by using a vacuum pump; (S2) injecting carrier gas into the powder chamber and mixing with hydroxyapatite powder; (S3) spraying the mixture of the powder and the carrier gas onto the surface of metal substrate in the deposition chamber through a nozzle and depositing hydroxyapatite coating layer on the metal substrate; (S4) hydrothermal-treating the deposited hydroxyapatite coating layer; and (S5) rinsing the hydrothermal-treated hydroxyapatite coating layer and drying the layer.

### BRIEF DESCRIPTION OF THE DRAWINGS

The above and/or other aspects of what is described herein will be more apparent by describing certain exemplary embodiments with reference to the accompanying drawings, in which:

FIG. 1 illustrates the XRD pattern of the powder of hydroxyapatite (HA) before deposition (a) and XRD pattern of hydroxyapatite coating deposited on the titanium substrate (b);

FIG. 2 illustrates the XRD patterns of hydroxyapatite coating deposited on a titanium substrate and hydrothermal-treated, in which pattern (a) represents embodiment 1, (b) is embodiment 2, (c) is embodiment 3, (d) is comparative example 1, and (e) is comparative example 2;

FIG. 3 illustrates FT-IR spectra of hydroxyapatite coating deposited on the titanium substrate in which spectrum (a) represents hydroxyapatite coating before hydrothermal treatment, (b) is embodiment 1, (c) is embodiment 2, (d) is embodiment 3, (e) is comparative example 1, and (f) is comparative example 2;

FIG. 4A illustrates Scanning Electron Microscopy (SEM) image of a surface hydroxyapatite coating deposited in titanium substrate; and FIG. 4B illustrates SEM image of the polished section.

FIG. 5A illustrates Transmission Electron Microscopy (TEM) image of hydroxyapatite coating before thermal treatment, FIG. 5B illustrates TEM of embodiment 3, FIG. 5C illustrates TEM of comparative example 1, and FIG. 5D illustrates TEM of comparative example 2;

FIG. 6 illustrates the average size of crystallites of hydroxyapatite within the hydroxyapatite coating according to the heating method and temperatures;

FIGS. 7A to 7E illustrate SEM images showing the coating state after 7 days of being dipped in Simulated Body Fluid (SBF) in which: FIG. 7A shows hydroxyapatite coating before thermal treatment, FIG. 7B shows embodiment 2, FIG. 7C shows embodiment 3, FIG. 7D shows comparative example 1, and FIG. 7E shows comparative example 2; and

FIG. 8 illustrates a graph showing alkaline phosphatase (ALP) activity of cells separated from each substrate.

### DETAILED DESCRIPTION OF EXEMPLARY EMBODIMENTS

The followings are detailed explanation of a preparation method of hydroxyapatite coating layer according to an embodiment. In one embodiment, a method of preparing a nano-structured hydroxyapatite coating layer with improved biocompatibility may include: (S1) putting hydroxyapatite [Ca₁₀(PO₄)₆(OH)₂, HA] powder in a powder chamber, placing a metal substrate in a deposition chamber, and keeping the chamber in vacuum state by using a vacuum pump; (S2) injecting carrier gas into the powder chamber and mixing with hydroxyapatite powder;

(S3) spraying the mixture of the powder and the carrier gas onto the surface of metal substrate in the deposition chamber through a nozzle and depositing hydroxyapatite coating layer on the metal substrate; (S4) hydrothermal-treating the deposited hydroxyapatite coating layer; and (S5) rinsing the hydrothermal-treated hydroxyapatite coating layer and drying the layer.

According to one embodiment, at S(1), hydroxyapatite [Ca₁₀(PO₄)₆(OH)₂, HA] powder is put into a powder chamber, a metal substrate is placed in a deposition chamber, and the chamber is kept in vacuum state using a vacuum pump.

It is desirable to use the hydroxyapatite powder obtained by heating under air pressure and between 1,000° C. and 1,200° C. Under 1,000° C., green compact forms during coating. Above 1,200° C., coating layer is not formed during coating and substrate is damaged.

In one embodiment, the metal substrate may be magnesium, titanium, titanium alloy, or magnesium alloy, although the metal substrates are not confined to those mentioned above. That is, any materials may be used if they can be used as bio metal substrates.

It is desirable to keep the vacuum level between 0.1 and 10 Torr by using a vacuum pump. The powders of raw materials are sprayed onto the metal substrate due to pressure difference between the powder chamber in air pressure and the deposition chamber in vacuum state.

At S(2), carrier gas is injected into the powder chamber and mixed with hydroxyapatite powder.

It is desirable to keep the amount of carrier gas between 5 and 50 L/min. If the amount is below 5 L/min, it is hard for the gas to be deposited on the substrate rapidly enough, in which case the coating layer cannot be formed with sufficient density and uniform coating layer, and strong adhesion between coating layer and the metal substrate is not achieved. If the amount of the carrier gas exceeds 50 L/min, an excessive amount of powder collides against the substrate, and flaws such as indentations may occur if big grains collide against the substrate. The carrier gas may be oxygen, nitrogen, helium, argon or air. As the type of the carrier gas does not affect the properties of the coating layer, air may desirably be used to reduce cost.

At S(3), the powder, mixed with carrier gas, is sprayed onto the surface of the metal substrate in deposition chamber through nozzle and hydroxyapatite coating layer is deposited on the metal substrate. Due to the pressure difference between two chambers, hydroxyapatite powder mixed with the carrier gas in the powder chamber at S(2) is sprayed onto the metal substrate of the deposition chamber through a slit type nozzle in which a rectangle hole is formed, forming a dense coating layer through the collisions of accelerated grains against the metal substrate.

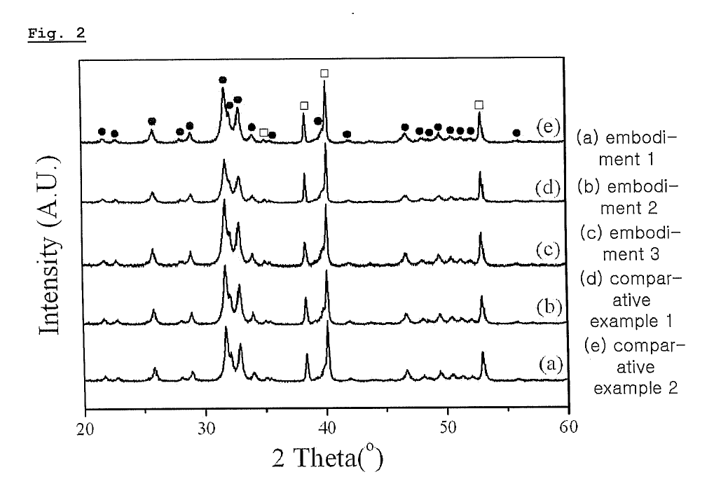

At S(4), the deposited hydroxyapatite coating layer of S(3) is hydrothermal-treated. FIG. 2 illustrates the XRD patterns of the hydrothermal-treated hydroxyapatite coating deposited on the metal substrate. Referring to FIG. 2, crystallinity of amorphous hydroxyapatite is increased by low-temperature hydrothermal process using distilled water as a gas source. Because the presence of hydroxyl expedites the crystallization of amorphous hydroxyapatite, crystallinity of coating layer improves greatly.

It is desirable to keep the heating temperature between 150° C. and 300° C. Below 150° C., the coating layer is not sufficiently crystalized. Above 300° C., biocompatibility is drastically dropped due to increased size of the grains within the coating layer.

The average crystallites size of grains within the hydroxyapatite coating layer obtained by the hydrothermal treatment may desirably be 50 nm or below, and more desirably, 30 nm or below. If the size exceeds 50 nm due to the growth of the grains within coating layer, biocompatibility of hydroxyapatite coating layer drastically drops. By hydrothermal treatment, the growth of the grains in the coating layer is inhibited greatly and the nano-size of the crystallites remains, which means the resultant layer is desirable for implant in view of its biocompatibility.

At S(5), the hydrothermal-treated hydroxyapatite coating layer is rinsed and dried. Rinsing may be carried out with distilled water and drying may be carried out in a traditional manner.

According to one embodiment, a nanostructured hydroxyapatite coating layer with improved biocompatibility prepared by the above-explained method is provided. The hydroxyapatite coating layer of the embodiment has superior adhesion to the metal substrate, high crystallinity, and superior biocompatibility due to the small grains size, and thus is suitable for use in orthopedics or dental and medicinal field.

### Fabrication of Hydroxyapatite (HA) Powder

Commercially available hydroxyapatite powder, with the average diameter (d₅₀) 15 nm per volume, was used as a starting coating powder. To acquire powder of suitable size of grains for AD, the hydroxyapatite powder was heated under the air pressure at 1050° C. for 2 hours.

### Embodiments 1˜3

Step 1—hydroxyapatite coating on a titanuim substrate using AD

Commercially available Ti substrate (10 mm×10 mm×0.5 mm) (CP-Ti, JIS grade 2) was scrubbed by 2000 grit SiC paper and washed in a ultrasonic wave with distilled water and acetone. After that, hydroxyapatite was deposited on the substrate using AD system in which hydroxyapatite powder was put into the powder chamber and Ti substrate was installed in the deposition chamber. After the deposition chamber was emptied by rotary vacuum pump connected to a pressuring pump machine, 30 L/min of air as carrier gas was flowed into the powder chamber. Due to the pressure difference between two chambers, hydroxyapatite powder mixed with the carrier gas from powder chamber was sprayed from deposition chamber onto the Ti substrate through the slit type nozzle with 10×0.5 mm² rectangle hole. Collision of accelerated grains against Ti substrate resulted in dense and uniform coating layer with 10 μm thickness.

### Step 2—Hydrothermal Treatment of Hydroxyapatite Coating Layer

Deposited hydroxyapatite coating layer was hydrothermal-treated by using polytetrafluoroethylene (Teflon) type stainless steel autoclave with 100 cm³ internal volume. 40 ml of distilled water was used as steam inside the autoclave. The autoclave was hydrothermal-treated at 150° C. (Embodiment 1), at 170° C. (Embodiment 2), and at 190° C. (Embodiment 3) for 20 hours, respectively. After hydrothermal-treating was completed, substrate with the coating layer formed thereon was washed with distilled water and dried at room temperature.

### Comparative Examples 1 and 2

Step 1—Hydroxyapatite coating on titanium substrate by AD

Commercially available pure Ti substrate of 10 mm×10 mm×0.5 mm (CP-Ti, JIS grade 2) was scrubbed by 2000 grit SiC paper and washed in a ultrasonic wave with distilled water and acetone. After that, in the same manner as in S(1) of Embodiment 1, hydroxyapatite was deposited on Ti substrate.

### Step 2—Heating Hydroxyapatite Coating Layer in Furnace

By using a traditional furnace, hydroxyapatite was heated at 400° C. (Comparative example 1) and at 500° C. (Comparative example 2) for 1 hour, respectively. The thermal treatment in furnace was set to be as low as 1.5° C./min to minimize the thermal stress caused by the thermal expansion difference between the coating layer and the substrate.

### Experiment 1

### Analysis of the Phase Evolution of Hydroxyapatite Coating Layer in Thermal Treatment

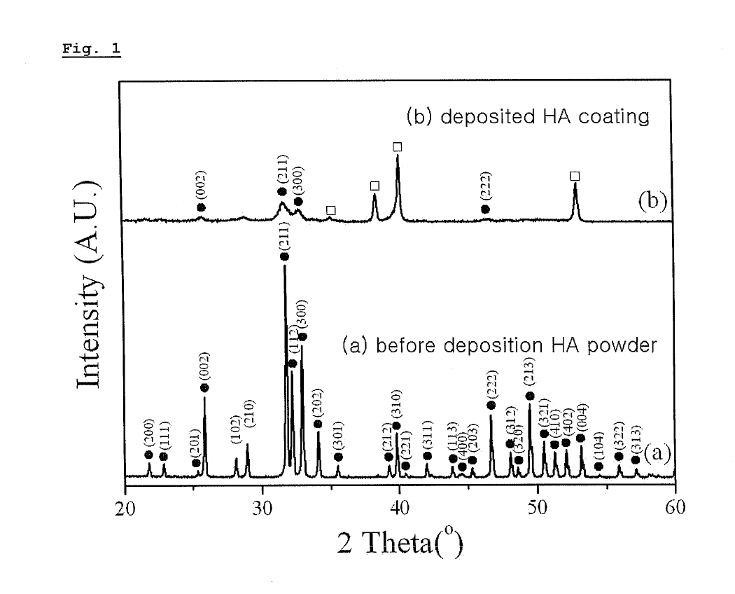

To analyze the phase evolution of hydroxyapatite coating layer in thermal treatment, X-ray diffractometer (XRD, X′pert MPD 3040, Philips Ltd., Eindhoven, Netherlands) was conducted with respect to the hydroxyapatite powder coating layer before deposition, and to the hydroxyapatite powder coating layer after deposition and before hydrothermal treatment respectively. The XRD data was acquired at 36 kV and 26 mA with single CuKa radiation and the result is illustrated in FIG. 1. In comparing waveforms (a) and (b) of FIG. 1, waveform (b) represents weaker and wider hydroxyapatite peak, indicating the low crystallinity of coating layer and small size of the crystallites.

Also, to compare the effects by the hydrothermal treatment and the traditional heating of the coating layer by furnace, X-ray diffractometer was conducted with respect to Embodiments 1 and 3 and Comparative examples 1 and 2 in the same manner as explained above, and the result is illustrated in FIG. 2. Referring to FIG. 2, the number and strength of peaks of the hydrothermal-treated hydroxyapatite is increased. In particular reference to Embodiments 1 and 3 where the heating temperature is below 300° C., the crystallinity of deposited hydroxyapatite coating layer is drastically improved. Therefore, hydrothermal treatment is proven to be an effective way to result in a low temperature crystallization of hydroxyapatite coating layer by AD.

### Experiment 2

### Observation of Structural Changes of Hydroxyapatite Coating Layer After Thermal Treatment

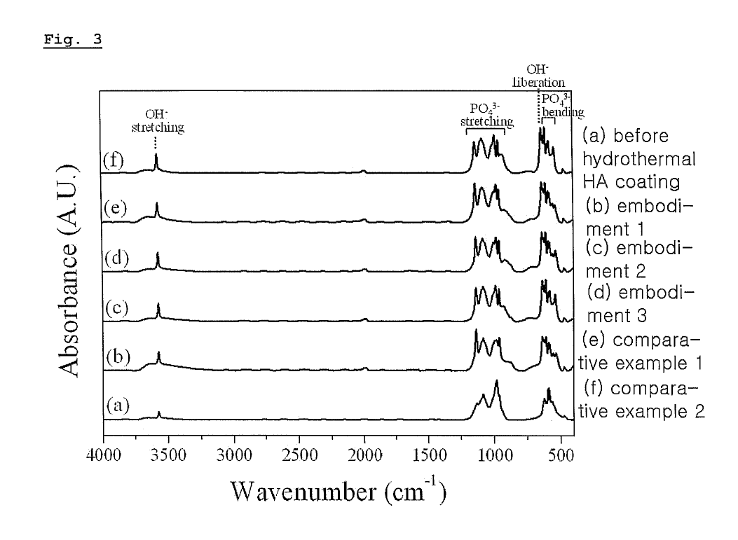

Structural changes of hydroxyapatite coating layer after thermal treatment were observed with the infrared spectrophotometer (FT-IR, IFS 66, Bruker Optics, Ettlingen, Germany) in the range of between 400 and 4,000 cm⁻¹. FIG. 3 illustrates the FT-IR spectra of hydroxyapatite coating deposited on Ti substrate. FIG. 3 shows that peaks of PO₄³⁻ and OH⁻ were distinct after hydrothermal treatments according to Embodiment 1 and 3 and Comparative example 1, 2. It shows that hydrothermal treatment has effect of improving crystallinity of coating layer, which corresponds with the result of X-ray diffractometer of Example 1.

### Experiment 3

### Observation of Micro Structure of Hydroxyapatite Coating Layer

Micro structure of hydroxyapatite coating layer was observed with Scanning Electron Microscope (SEM, JSM-5800, Jeol Co., Tokyo, Japan) and Transmission Electron Microscope (TEM, JEM-2100F, Jeol Co., Japan).

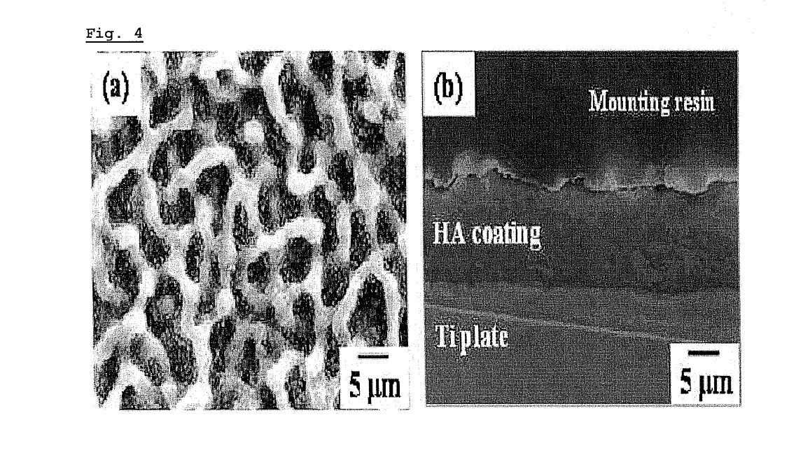

FIG. 4A illustrates the surface shape of SEM image of the hydroxyapatite coating layer prepared according to Embodiment 1. The hydroxyapatite coating layer shows the coarse surface with network-type micro-structure. The coarse surface of hydroxyapatite coating layer provides better environment for osteoblast in attachment, proliferation and segmentation. FIG. 4B illustrates the SEM cross sectional image of the hydroxyapatite coating layer prepared by the method of Embodiment 1. FIG. 4(b) shows that the interface between coating layer and Ti substrate is continuous without pore or defect. Therefore, hydroxyapatite coating layer according to the invention is expected to have superior biocompatibility and strong adhesion between coating layer and metal substrate.

### Experiment 4

### Measurement of the Crystallite Size of the Hydroxyapatite

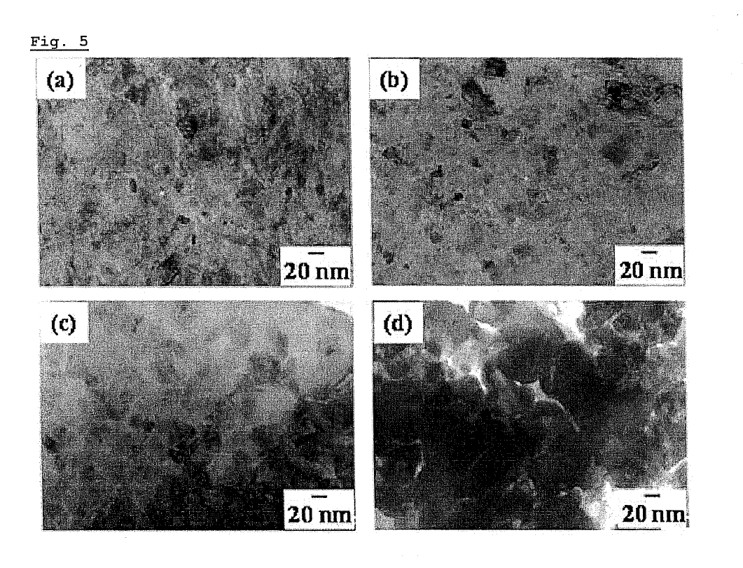

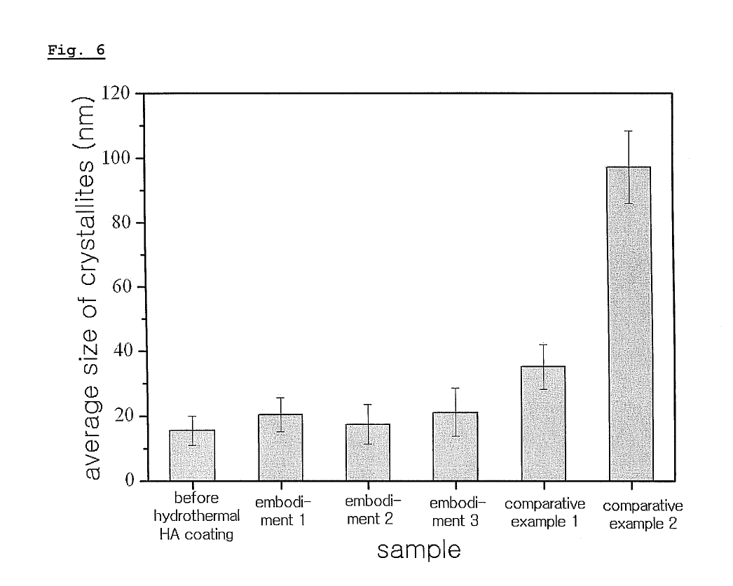

The crystallite size of hydroxyapatite coating is measured based on TEM photos by using image analysis software (Image-Pro, version 4.0, Media Cybernetics, L.D., silver spring, Md., U.S.A.) which is commercially available. FIG. 5A to 5D illustrate the hydroxyapatite coating layers each of which being before hydrothermal treatment; by Embodiment 3; and by Comparative examples 1 and 2, observed by Transmission Electron Microscope (TEM). FIG. 6 illustrates the average size of crystallites measured by above image anaylsis software.

Referring to FIGS. 5A to 5D, the coating layer before the thermal treatment and the coating layer by Embodiment 3 have a relatively small hydroxyapatite grain size while the coating layers of Comparative examples 1 and 2 have a growth of the grains into bigger size with thermal treatment, and thus have a considerably big grains. The above is more obvious from FIG. 6. The hydroxyapatite coating layer prepared by Embodiments 1 and 3 and the hydroxyapatite coating layer without thermal treatment have the average size of grains between 17.4 and 21.1 nm. On the other hand, in Comparative examples 1 and 2 where the heating is carried out in a traditional manner between 400° C. and 500° C., the average size of grains grows up to 97.3 nm.

### Experiment 5

### Evaluation of Bioactivity

When bio-metal materials coated with hydroxyapatite is implanted in the human body, hydroxyapatite crystallites are formed in the coating layer, and bio-metal materials are attached to the bone, or the like of the body due to the crystallites. Therefore, the bioactivity level of metal materials can be evaluated by the speed of crystallites forming in the simulated body fluid (SBF).

To confirm the bioactivity of a metal substrate coated with hydroxyapatite, the following experiment was carried out.

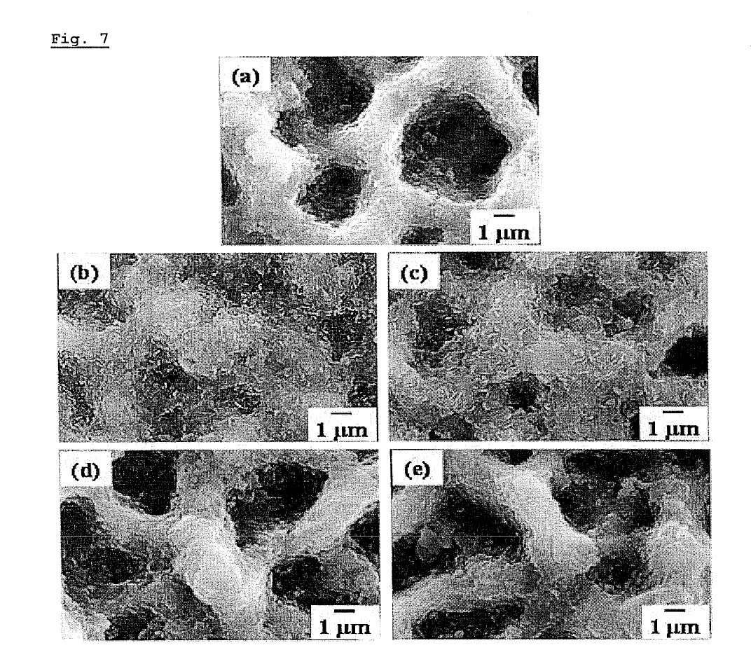

Ti substrate on which hydroxyapatite coating layer is deposited without thermal treatment, and Ti substrates of Embodiments 2 and 3 and Comparative examples 1 and 2 were soaked in simulated body fluid (SBF) for 7 days. FIG. 7 illustrates the SEM image of micro structure of each surface of Ti substrate after the 7 days. FIGS. 7A to 7E are SEM images of nano-structures of the respective surfaces. Referring to FIGS. 7A to 7E, Ti substrate with no thermal treatment and Ti substrates of Comparative examples 1 and 2 show meager changes in the nano-structure of the surface, while, on the surfaces of Ti substrates of Embodiments 2 and 3, newly precipitated crystallites are evenly distributed. This is because the hydroxyapatite coating layers of Embodiments 2 and 3 were thermal-treated at a relatively low temperature and thus have smaller sizes of grains within coating layer compared to those of Comparative examples 1 and 2. Therefore, it is obvious that hydroxyapatite coating layer by hydrothermal treatment according to an embodiment has a superior bioactivity.

### Experiment 6

### Evaluation of Biocompatibility

To confirm the biocompatibility of a metal substrate with a hydroxyapatite coating layer thereon, the following experiment were conducted.

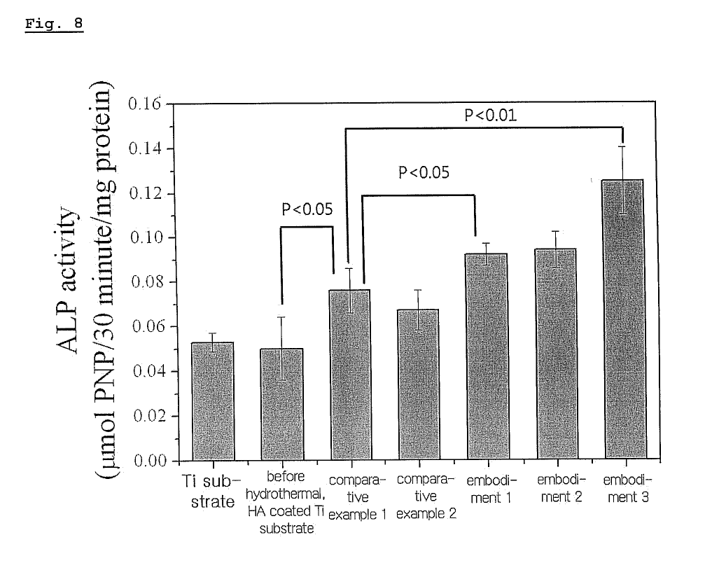

To confirm the biocompatibility of the Ti substrate, Ti-substrate, Ti-substrate with non-hydrothermal-treated hydroxyapatite deposited thereon prepared by step 1 of Embodiment 1, substrates prepared by Embodiments 1, 2 and 3, substrates prepared by Comparative examples 1 and 2 were seeded with MC3T3-E1 preosteoblast cells (CRL-2593, ATCC, Manassas, Va., USA), with cell density of 1×10⁴ cell/ml, and were cultured for 10 days. After 10 days, each cell-seeded substrate was washed with phosphate buffered saline (PBS) and each cell was separated from each substrate with trypsin ethylene diamine tetraacetic acid (Trypsin-EDTA). ALP activity levels of the separated cells were measured and p-nitrophenyl phosphate (ELISA, Sigma, St. Louis, Mo., USA) was used as ALP substrate. The absorbance of reaction product, p-nitrophenyl was measured with microplate reader in 405 nm, and its result is illustrated in FIG. 8.

Referring to FIG. 8, the Comparative example shows superior ALP activity level compared to the substrate before hydrothermal-treatment. Further, the Embodiments show superior ALP activity level compared to the Comparative examples. Therefore, it is inferable that the substrate with a hydroxyapatite coating thereon and treated hydrothermal has the excellent biocompatibility, and this is due to the nano-structured hydroxyapatite coating layer and its high crystallinity according to an embodiment.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A preparation method of a nano-structured hydroxyapatite coating layer with improved biocompatibility, which comprises:
(S1) putting hydroxyapatite [Ca₁₀(PO₄)₆(OH)₂, HA] powder in a powder chamber, placing a metal substrate in a deposition chamber, and keeping the chamber in vacuum state by using a vacuum pump;
(S2) injecting carrier gas into the powder chamber and mixing with the hydroxyapatite powder;
(S3) spraying the mixture of the powder and the carrier gas onto the surface of a metal substrate in the deposition chamber through a nozzle and depositing the hydroxyapatite coating layer on the metal substrate;
(S4) hydrothermal-treating the deposited hydroxyapatite coating layer, and
(S5) rinsing the hydrothermal-treated hydroxyapatite coating layer and drying the layer.

2. The method of claim 1, wherein the hydroxyapatite powder of S(1) is obtained with 1-3 hours of thermal-treatment at 1000 to 1200° C. under air pressure.

3. The method of claim 1, wherein the metal substrate of S(1) may be selected from a group consisting of: magnesium, titanium, magnesium alloy, titanium alloy.

4. The method of claim 1, wherein the temperature of hydrothermal treatment of S(4) is between 150 and 300° C. .

5. The method of claim 1, wherein the average crystallites size of the hydroxyapatite deposition hydrothermal-treated at S(4) is below 50 nm.

6. The method of claim 5, wherein the average size of crystallites of the hydroxyapatite deposition hydrothermal-treated at S(4) is below 30 nm.

7. A nano-structured hydroxyapatitie coating layer with improved biocompatibility, fabricated according to claim 1, wherein hydroxyapatite is deposited on the surface of a titanium substrate.
